(19) United States
(12) Patent Application Publication
(10) Pub. No.: US 2019/0105450 A1
(43) Pub. Date: Apr. 11, 2019

(54) ADHERENCE MONITOR FOR A MEDICAMENT INHALER

(71) Applicant: ADHERIUM (NZ) LIMITED, Auckland (NZ)

(72) Inventors: Garth Campbell SUTHERLAND, Auckland (NZ); Michael James GORMACK, Auckland (NZ)

(73) Assignee: ADHERIUM (NZ) LIMITED, Auckland (NZ)

(21) Appl. No.: 15/763,338

(22) PCT Filed: Sep. 26, 2016
(86) PCT No.: PCT/IB2016/055726

§ 371 Date: Mar. 26, 2018

(30) Foreign Application Priority Data
Sep. 25, 2015 (NZ) 712748

(51) Int. Cl. A61M 15/00; A61B 5/00

(52) U.S. Cl. CPC A61M 15/008; A61M 15/009; A61B 5/4833; A61M 15/0026; A61M 2205/50; G16H 20/10; A61M 2205/3375; A61M 2205/3368; A61M 2205/332; A61M 2205/587; A61M 2205/18; A61M 2205/3584

(57) ABSTRACT

Some embodiments are directed to an adherence monitor for a medicament inhaler and methods for detecting cap removal. Two sensors are provided to detect cap removal/presence, in order to reduce or minimise risk of inadvertent sensing of cap removal.

### CROSS REFERENCE TO RELATED APPLICATIONS

This application is a national phase filing under 35 C.F.R. § 371 of and claims priority to PCT Patent Application No. PCT/162016/055726, filed on Sep. 26, 2016, which claims the priority benefit under 35 U.S.C. § 119 of New Zealand Patent Application No. 712748, filed on Sep. 25, 2015, the contents of each of which are hereby incorporated in their entireties by reference.

### BACKGROUND

Some embodiments relate to methods, devices and systems for monitoring adherence to medication regimes for inhalers with a mouthpiece cover.

Some embodiments are directed to adherence monitors for inhalers with a mouthpiece cap or cover. These are often used in the treatment of respiratory diseases such as asthma, COPD, cystic fibrosis, and bronchiectasis. However, such devices may also be used to deliver other medications, for example for the treatment of pain, heart conditions, erectile dysfunction, diabetes, and other indications. The general term cap or cover will be used interchangeably to refer to all such covers and caps, however constructed, which serve to close or protect the mouthpiece when it is not in use, and are opened or removed when the inhaler is to be used.

A common type of medicament inhaler is a pressurised metered dose inhaler (pMDI). Such inhalers generally include a medicament canister and an actuator. The medicament canister contains medicament under pressure and is designed to deliver a metered dose of medicament in the form of an aerosol spray. The actuator includes a generally L-shaped hollow tube which has a first open end adapted to receive the medicament canister, and a second open end which acts as a mouthpiece. The mouthpiece is usually fitted with a removable cap, which may be tethered to the inhaler or separate from it.

There are several different types of pMDI inhalers with tethered cap mouthpiece cover available on the market. In the USA, examples of the inhalers include: Ventolin® HFA (GSK); Advair® (GSK); Flovent® (GSK); Duolin® (Cipla); Symbicort® (AstraZeneca) among others.

Another type of medicament inhaler is a dry powder inhaler (DPI). A common type of dry powder inhaler is in the form of a generally tube-shaped body (e.g. a TURBUHALER® which is manufactured and marketed by AstraZeneca AB), which includes an internal store of a suitable medicament; a rotatable base for dispensing a single dose of the medicament into an appropriate inhalation chamber; and a mouthpiece, through which a user may inhale the medicament that has been dispensed into the inhalation chamber. Such dry powder inhalers usually come with a removable and replaceable screw-cap, adapted to cover the mouthpiece and tube-shaped body of the inhaler, when the inhaler is not in use.

Another common type of a DPI is in the form of a disc (e.g. GSK's Diskus® inhaler) which includes a priming lever, and the priming lever, when actuated, dispenses a metered dose of medicament in the form of a dry powder into an appropriate receptacle adjacent a mouthpiece (which is usually covered by a cap when the DPI is not being used). The dry powder may then be inhaled by the user (namely, by sucking strongly on the mouthpiece of the inhaler).

Another type of medicament inhaler is a breath-actuated inhaler (BAI). A BAI is in the form of a pMDI or a DPI in which the dose is delivered by a triggering mechanism internal to the inhaler in response to inspiratory flow rates exceeding certain pre-set levels, i.e. a patient's inhalation causes the dose to be delivered. An example of such a pMDI BAI is the Easi-Breathe® which is manufactured and marketed by Ivax/Teva.

Another type of DPI is an inhaler in which the medicament is held within a capsule which is perforated by the user (e.g. by a use of a piercing button on the inhaler) prior to the inhalation during which the medicament is delivered (e.g. HandiHaler® manufactured and marketed by Boehringer Ingelheim Pharma GmbH & Co. KG or Breezhaler® marketed by Novartis. Other types of DPI are also known (e.g. Genuair® by Almirall).

Another type of DPI inhaler is ELLIPTA® (manufactured and marketed by GSK), and sold under a number of brand names: ANOROCELLI PTA®, BREO®ELLI PTA®, INCRUSEC®ELLIPTA®, RELVAR®ELLIPTA®. The ELLIPTA® DPI is described in a number of granted patents and patent applications, e.g. U.S. Pat. Nos. 8,161,968 and 8,746,242.

A number of the breath-actuated or dry powder inhalers include hinged mouthpiece covers.

### SUMMARY

A problem associated with the use of all medicament inhalers is poor adherence. Many studies have shown that users frequently do not take their medicament at the predetermined or prescribed times and/or in the required amounts. The consequences of this non-adherence can include reduced disease control, lower quality of life, lost productivity, hospitalisation and avoidable deaths. This represents a considerable cost to the users, as well as to the health system.

To address this problem, some of the inhalers incorporate dose counting mechanism to indicate the number of medicament doses delivered and/or the number of medicament doses remaining in the medicament inhaler. However, a limitation associated with early (mechanical) dose counters is that they were limited to just recording the number of doses only, that is, no other compliance data was gathered.

Adherence monitoring devices have been developed for use with medicament inhalers allowing for wider range of inhaler use data to be gathered and available to patients and healthcare providers remotely.

Many inhalers include a cap over the mouthpiece. The cap ensures that the mouthpiece remains clean, that no foreign objects can enter the mouthpiece. An issue associated with the use of all inhalers equipped with a cap is that users sometimes inadvertently administer medication (e.g. by pushing down the canister of a pMDI) without removing the cap from the inhaler. In such instance an adherence monitoring device may still register a dose as delivered and not recognise that the canister actuation occurred when the cap was still on the mouthpiece.

The ability to record compliance data relating to when the cap is removed and replaced, and/or how many times the user dispenses (or attempts to dispense) a dose of medicament, with the cap still attached to the mouthpiece, would provide very useful and important information, both for training purposes or feedback for the user, as well as for general medicament compliance data gathering purposes.

Patent application WO/2015/030610 by the present applicant discloses a compliance monitor for a medicament delivery device capable of detecting the presence or absence of a tethered cap on the mouthpiece and distinguishing between dose deliveries which occur with cap on vs cap off. In one embodiment described, the presence or absence of cap is detected via a single switch located at the base of the adherence monitoring device.

Patent applications WO/2015/133909 and WO/2016/043601 by the present applicant disclose compliance monitors for dry powder inhalers capable of detecting the presence or absence of a mouthpiece cap. In some embodiments described, the presence or absence of a cap is detected via a lever switch extending generally parallel to the axis of rotation of the inhaler base or through engagement between the cap, sliding clip and a lever switch located in the compliance monitor.

Patent application WO/2016/111633 by the present applicant discloses compliance monitors for BAI and DPI inhalers capable of detecting presence or absence of a hinged mouthpiece cover.

All above solutions rely on a single sensor based detection of the removal or replacement of the mouthpiece cover.

Whilst this approach is effective, another problem arises. In some instances the single switch can be inadvertently triggered by the user. For example, if the user accidently presses on the switch during a valid dose delivery, the adherence monitoring device may register that the cap is on and log the dose delivery as an invalid dose. In such instance the adherence data for the patient would incorrectly show decreased medication compliance.

Some embodiments are therefore directed to an enhanced or an improved adherence monitor for inhalers equipped with cap which is able to provide more reliable cap status data.

In a first broad form, some embodiments are directed to an adherence device with a two sensor system, in which both sensors must be triggered in order to register as a cap removal event.

According to one aspect, some embodiments provide an adherence monitor for a medicament inhaler, the inhaler including a body, a mouthpiece for operatively inhaling the medicament, a cap for the mouthpiece, wherein, the monitor includes at least two sensors associated with the cap, so that removal of the cap triggers both of the sensors.

According to another aspect, some embodiments provide a method for detecting the removal of a cap from a medicament inhaler, the medicament inhaler including at least a body, a mouthpiece for operatively inhaling the medicament, and a cap for the mouthpiece, the method including:

providing at least two sensors, each arranged to be triggered by removal of the cap;
determining at a control device whether both sensors have been triggered; and
if both of the sensors have been triggered, thereby determining that a cap removal event has occurred.

In another aspect, some embodiments provide an adherence device with a two sensor system, in which at least one sensor must be triggered by operation of the tether in order to register a cap removal event.

According to a further aspect, some embodiments provide an adherence monitor for a medicament inhaler, the inhaler including a body, a mouthpiece for operatively inhaling the medicament, a cap for the mouthpiece, and a tether connecting the cap to the body, the monitor including at least two sensors associated with the tether, so that removal of the cap triggers both of the sensors.

According to another aspect, some embodiments provide a method for detecting the removal of a cap from a medicament inhaler, the inhaler including a body, a mouthpiece for operatively inhaling the medicament, a cap for the mouthpiece, and a tether connecting the cap to the body, the method including:

providing at least two sensors, at least one sensor associated with the tether;

determining whether both sensors have been triggered; and

if so, thereby determining that the cap has been removed.

The provision of two switches greatly reduces the risk that an inadvertent action by the user will trigger a cap off status indication, as it is significantly more unlikely that two switches will be mistakenly operated. This provides significant advantages in data reliability in suitable implementations.

### BRIEF DESCRIPTION OF THE DRAWINGS

An illustrative embodiment will be described with reference to the accompanying figures, in which:

FIG. 1 is a perspective view of a first embodiment attached to a Symbicort® inhaler;

FIG. 2 is a perspective view of the embodiment illustrated in FIG. 1 without the inhaler and with the hinged door of the adherence monitor opened;

FIG. 3 is an inverted perspective view of another embodiment, with the inhaler in and the cap covering the inhaler mouthpiece;

FIG. 4 is an inverted perspective view of the embodiment illustrated in FIG. 3, with the inhaler in and the cap and tether open;

FIG. 5 is a bottom view of the embodiment illustrated in FIG. 2;

FIG. 6 is a perspective view of another embodiment, where the sensors are located on the related art adherence monitor designed for use with HandiHaler® inhaler;

FIG. 7 is a perspective view of another embodiment, showing alternative locations of sensors on an adherence monitor designed for use with a pMDI fitted with a non-tethered cap; and

FIG. 8 is a perspective view of another embodiment, wherein the adherence monitor is designed to fit Symbicort®.

### DETAILED DESCRIPTION OF EXEMPLARY EMBODIMENTS

Throughout this specification, the terms “patient” or “user” or “person” or “patient usage”, when used in relation to the use of a medicament delivery device, are to be understood to refer to any person that uses a medicament delivery device.

Some embodiments will be described in relation to various specific implementations, which it will be understood are intended to be illustrative and not limitative of the scope of some embodiments. It will be appreciated in particular that various additional features and functions, indicators and the like may be included in monitors which implement some embodiments. These may be selected for specific application at the option of the product designer.

The following implementations will be described with reference primarily to pressurized metered dose inhalers (pMDI) and dry powder inhalers (DPIs), as these are in widespread commercial use. However, some embodiments may, with suitable modifications as will be apparent to those of ordinary skill in the art, be applied to other designs of inhalers using mouthpiece covers, presently known or yet to be developed. The general term inhaler will be used to refer to any such inhaler device, unless a contrary intention is apparent from the context.

Similarly, whilst the discussion below is principally in relation to respiratory related medicaments, it is applicable to any use of medication dispensing inhaler devices, including, by way of example only, pain medication, diabetes, erectile dysfunction, or other conditions. Some embodiments are concerned with the monitoring of how the medication is used and dispensed, and should in no way be considered as limited to any particular medicament or condition. The terms “medicaments” and “medication” should be broadly construed, and are not limited to any specific indication or types of inhalable substances.

Some embodiments are concerned with inhaler devices which incorporate a cover or cap. The function of the cap or cover is to impede or prevent foreign bodies or contamination from entering the device or affecting the mouthpiece when it is not in use. In some forms the cap is removable, for example by unscrewing or removing a force fit cap. In other forms that cap is tethered to the inhaler. In yet other forms of inhaler, the cover may be hinged, pivoting or otherwise moving to take the inhaler from an inoperative to an operative state. The general term cap or cover will be used to refer to all such covers and caps, however constructed, which serve to close or protect the mouthpiece when it is not is use, and are opened or removed when the inhaler is to be used. Similarly, the term “cap removal” refers to any such operation to remove the cap or cover or otherwise ready the inhaler for use, and cap replacement refers to replacing the cap or closing the cover or otherwise placing the inhaler into its inoperative condition.

It will be appreciated that some embodiments will be described with reference to implementations which are intended to be supplied as a device to be used over many replacement inhalers, attaching to one and then removing when the inhaler is no longer used, for attachment to a new inhaler. It could be sold as part of or attached to an inhaler and removed for attachment to another inhaler. However, some embodiments may also be implemented as an integral part of an inhaler. It could be sold as an inhaler with an embedded adherence monitor and disposed together with the inhaler device.

As a general explanation, the implementations of an adherence monitor described are intended to be used with a medicament dispensing inhaler. The devices include systems to detect that a dose has been dispensed, and to retain or communicate a record of this to a remote system, for example via Bluetooth® to a smartphone, tablet or other device. The intention is to automatically create a record of usage, to assist in clinical management and to provide remote user interface to enable patients to better manage their medication compliance. The devices may also provide reminders to the user, detect whether or not a dispensing device is attached, provide error indications, or provide other functions.

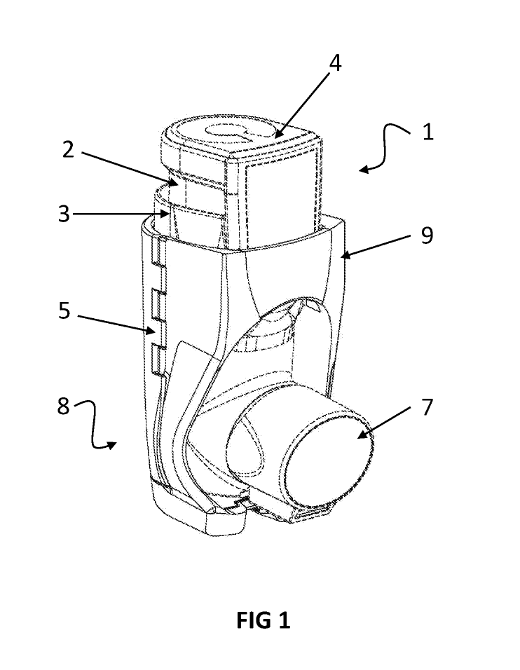

FIG. 1 shows a related art pMDI medicament inhaler (Symbicort® by AstraZeneca) generally indicated by arrow 1. The medicament inhaler 1 includes a store of medicament in the form of a pressurised medicament canister 2, and a first housing (for housing the canister 2) in the form of an actuator 3. The medicament inhaler includes a top counter 4 which when pressed down, pushes the canister 2 into actuation position.

The inhaler 1 is also provided with a medicament dispenser for delivering a dose of medicament. The medicament dispenser is in the form of a spray stem (not shown) extending from the canister 2, which is adapted to engage with a spray-directing element (not shown) housed within the actuator 3. When the canister 2 is pushed down into the actuator 3, the spray stem and spray-directing element combine to deliver a metered dose of medicament out through the mouthpiece 6 of the actuator, and into the mouth of the user (who sucks on the mouthpiece 6 at the same time as the medicament is dispensed).

The inhaler 1 is also provided with a removable and replaceable cap 7, which is adapted to close off the mouthpiece 6 when the inhaler 1 is not in use. The cap 7 ensures that the mouthpiece 6 remains clean, and free from dust and grime, and also ensures that no foreign objects (such as coins) can enter the mouthpiece 6 (for example when the inhaler is not in use or being carried in a pocket or purse), which may otherwise present a choking hazard.

The cap 7 is attached to the rear of the actuator 3 by a tether 15. Having the cap 7 tethered to the actuator 3 in such a fashion ensures that the cap 7 is not inadvertently dropped or lost once it has been removed. Furthermore, having a tethered cap 7 ensures that the cap 7 does not become a choking hazard (that is, the cap 7 could not inadvertently be swallowed by a user because it is attached to the actuator 3, via the tether 15).

Some embodiments may be applied with any suitable type of tether. As will be understood by those of ordinary skill in the art, the details of the engagement between the tether and the cap will vary, and will be selected by the manufacturer. Suitable tethers may be made using flexible or rigid materials. Materials for a rigid plastic tether may include ABS, PP, LDPE, or Tritan. Materials for a flexible tether may include urethane or rubber.

The adherence monitor 8 according to this implementation is housed within a second housing 9, which is releasably attachable to the inhaler 1 (or more specifically to the actuator 3).

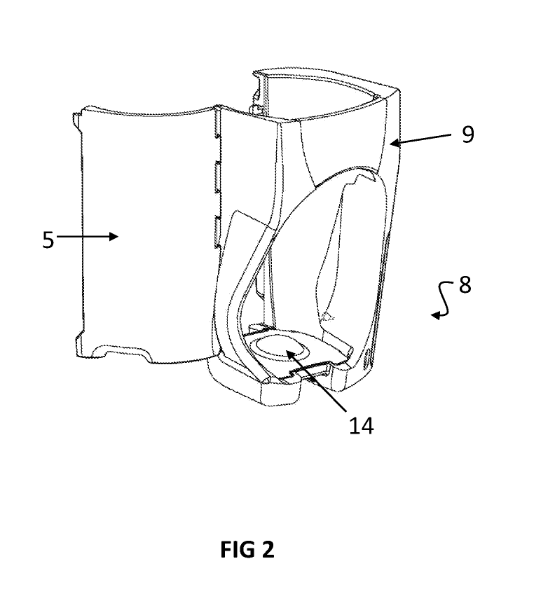

Referring to FIG. 2, the second housing 9 only partially encloses the inhaler 1. That is, the second housing 9 encloses the sides and front of the inhaler 1, and is fitted with a hinged door 5 which covers an opening at the rear of the second housing 9. This rear opening allows for the inhaler 1 to be placed into, secured and/or removed from, the second housing 9.

The adherence monitor 8 includes an electronics control module (ECM or processor, not shown) which is included within either a side or the base of the second housing 9. The ECM is adapted to monitor and/or manipulate and/or store and/or transmit compliance data relating to patient usage of the inhaler 1.

Because the second housing 9 is releasably attachable to the inhaler 1, it may be appreciated that the adherence monitor 8 may be portable and/or reusable across a range of different medicament inhalers.

The adherence monitor 8 includes a dose detector in the form of a first electromechanical switch 14, which is in electronic communication with the ECM. The first switch 14 is located on a lower internal surface of the second housing 9, and the first switch 14 therefore abuts the bottom of the actuator 3.

When a dose of medicament is dispensed, the canister 2 is pushed down into the actuator 3, as described previously. This pressure forces the bottom of the actuator 3 against the first switch 14, which closes (or actuates) the first switch 14, and thus the dispensing of the dose is detected, and an appropriate signal is sent to the ECM, where the dispensing of the dose is recorded, and the date and time of the dispensing of the dose is also recorded. Such a dose detecting apparatus has been previously described in patent application WO2013/043063 by the present applicant, which is incorporated herein, in its entirety, by reference.

The adherence monitor 8 also includes a cap detection system, as will be described below.

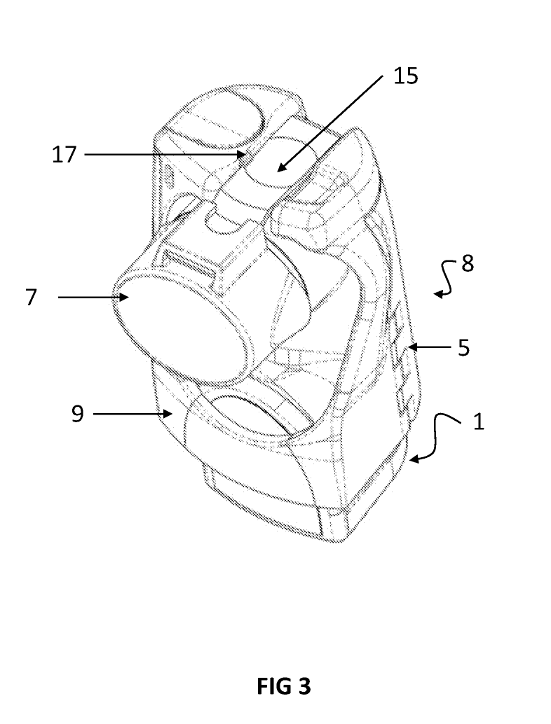

Referring to FIGS. 3, 4 and 5, the cap detection system of the present implementation is shown. The cap detection system includes two cap detection switches: switch 16 and switch 16′. The switches 16 and 16′ are located on the underside of the lower surface of the second housing 9 (that is, the underside of the adherence monitor 8). The switches are offset in such a way that a user's finger cannot reasonably press both buttons simultaneously when they are activating the inhaler, thereby preventing false triggering. Further, the switches 16 and 16′ are positioned so that when the cap 7 is on the mouthpiece 6, the tether 15 engages both switches 16 and 16′. The action of removing and replacing the cap 7 (with respect to the mouthpiece 6 of the inhaler 1) respectively disengages and engages both the switches 16 and 16′. To register a valid cap on state both switch 16 and 16′ must be closed.

It will be appreciated that in most situations, the removal of the cap will more or less simultaneously trigger operation of switches 16 and 16′. If the sensors are triggered, for example, several seconds apart, this is then likely to be simply some mishandling by the user, and the cap will still be off. The sensors should be triggered within a short time frame, to validly record an event, and this time period may be controlled in suitable implementations.

Hence, the action of removing and/or replacing the cap 7 results in an actuation (or de-actuation) of switches 16 and 16′, and an appropriate electrical signal is sent to the ECM. The cap detection system of some embodiments includes the cap detection sensors and the ECM.

If, at the same time as removing or replacing the cap 7, the user inadvertently dispenses a dose of medicament, the cap detection and the dose detection signals will be sent to the ECM. The ECM will, upon receiving both signals (more or less simultaneously) be able to determine that a dose of medicament has not in fact been dispensed to the user, and that instead a user error has occurred, and this determination (or the corresponding data, for analysis remotely) may be stored and/or transmitted, as described previously.

Hence, an appropriate electrical signal may be sent to the ECM indicating that the cap 7 is attached to the mouthpiece 6. If the user dispenses, or attempts to dispense, a dose of medicament whilst the cap 7 is attached to the mouthpiece 6, the cap detection system and the dose detection system will combine to detect this, and appropriate electrical signals will be sent to the ECM. The ECM, will thus be able to determine that the dose was dispensed (or attempted to be dispensed) with the cap 7 attached, and this determination may be stored and/or transmitted, as described previously.

Alternatively, and/or additionally, the user may be alerted to either of the above two errors by an indicator, substantially as described previously (indicator not shown in the drawings).

In such a fashion, it may be appreciated that compliance data relating to two common user errors of medicament inhalers may be collected, stored and/or used to provide feedback to the user regarding their techniques, and/or prompt a health professional to provide or schedule further training for the user in relation to their use of medicament inhaler 1.

Furthermore, either the user or a health care professional may be alerted to the fact that the user has not taken their medicament, even though a dose has been dispensed. This is an important consideration for a user who thought that they did in fact take their medicament (in the situation where the cap 7 is left on during the administering of a dose of medicament), and so that person can be alerted to take another dose (with the cap 7 removed). Hence, as well as ensuring proper medicament compliance generally, the adherence monitor 8 may also potentially prevent an exacerbation event.

It is fairly common for users to either inadvertently leave the cap 7 on when administering a dose of medicament and/or inadvertently dispense a dose of medicament when removing or replacing the cap 7, and these type of errors have potentially adverse consequences. For example, the patient may become ill or incapacitated (or worse) as a result of not having received their medicament at the required time.

Furthermore, a health care professional upon reviewing skewed compliance data (that is, data supplied by an inhaler that does not utilise the adherence monitor 8) may change the dosage regime that the user is currently on, without releasing that not as many doses of medicament have been taken as was thought. This scenario is clearly undesirable, and the adherence monitor 8 therefore serves as a very useful and important tool to address these type of issues.

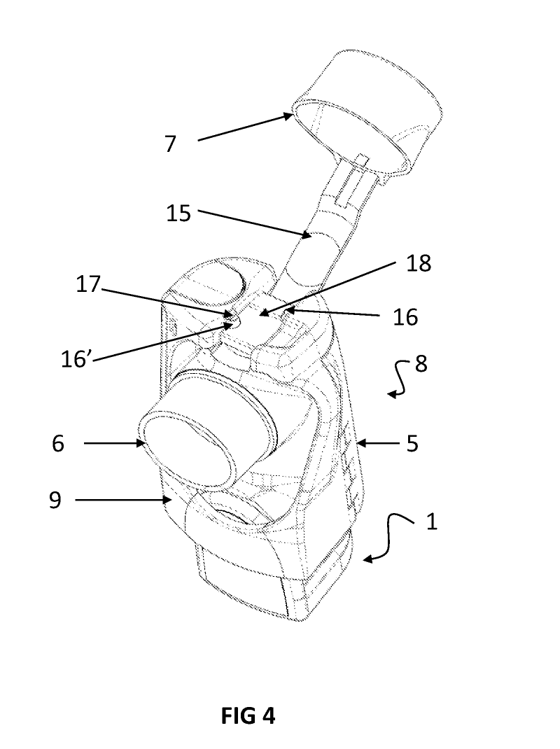

Having regard to FIGS. 3 and 4, there is shown an inverted view of another embodiment of the adherence monitor 8. In FIG. 3, the cap 7 covers the inhaler mouthpiece 6 and tether 15 engages switches 16 and 16′ (not shown). To ensure a firm fit, the tether 15 is further secured by catch 17. The tether 15 fits into the recess 18 configured to receive the tether 15 and included in the second housing 9. The arrangement shown in FIG. 3 is logged by the ECM as a ‘cap on’ status and any delivery of a medicament dose is logged as an invalid dose and recorded as such in the adherence monitor 8 memory. In FIG. 4, the cap 7 is removed from the mouthpiece 6 and the switches 16 and 16′ are no longer engaged by tether 15. The ECM detects the change in both the switch 16 and 16′ status and logs a ‘cap off’ event. A delivery of medication during a cap off event is logged as a valid medication delivery, subject to any additional confirmations of valid dose delivery obtained from other possible dose delivery sensors (e.g. acoustic sensors, motion sensors, temperature sensors, etc.).

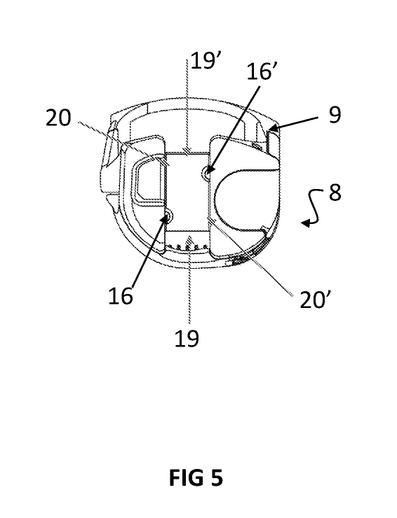

Having regard to FIG. 5 there is shown a bottom view of the adherence monitor 8 illustrated in FIGS. 1 to 2, with additional alternative positions for the switches illustrated by arrows 19 and 19′ and 20 and 20′. While the placement of sensors in symmetrical locations may be preferred, it is not necessary.

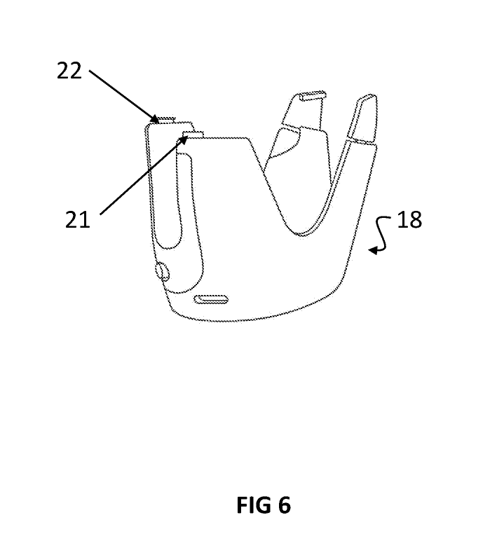

Referring to FIGS. 6, 7 and 8, there are shown in general terms further embodiments, where implementations of some embodiments using two-sensor systems of cap detection are shown in relation to other types of adherence monitors.

FIG. 6 illustrates a perspective view of another embodiment. The adherence monitor 18, described in the patent application by the present applicant (WO/2016/111633), is designed to monitor the usage of HandiHaler® inhaler. HandiHaler® (not shown) includes a hinged mouthpiece cover. Arrows 21 and 22 show one example of where the sensors can be located on the adherence monitor 18.

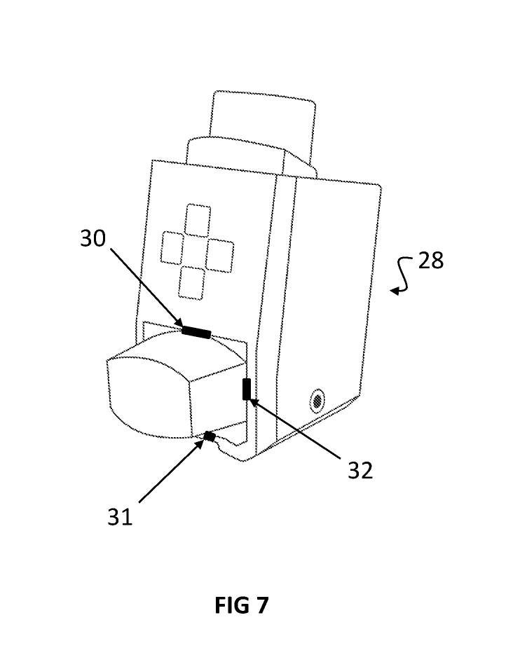

Referring to FIG. 7, there is shown an adherence monitor 28 designed for use with a pMDI fitted with a non-tethered cap. Arrows 30, 31 and 32 point to the possible locations of the sensors on the adherence monitor 28.

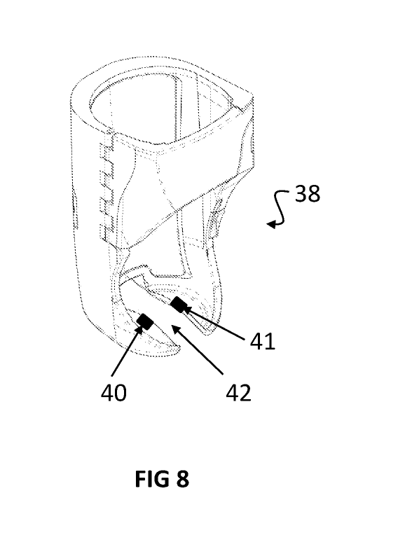

Having regards to FIG. 8, there is shown another embodiment. In this embodiment, the adherence monitor 38 is designed for use with Symbicort® inhaler. Symbicort® pMDIs (not shown) are generally fitted with a tethered cap. To accommodate the tether, the adherence monitor 8 is equipped with a tether opening 42. The cap removal sensors can be located along the inner edges of the tether opening 42 of the adherence monitor 38. Arrows 40 and 41 indicate one of many possible locations of the sensors.

Further, the two-sensor system of cap removal detection could be incorporated into other designs of adherence monitors, for example, adherence monitors described in WO/2015/133909, WO/2016/043601, WO/2106/111633, US2014/0000598 or US 2014/0182584 by the present applicant, the disclosures of which are hereby incorporated by reference.

Of course, it will be appreciated that the switches could be operated in a different mode than described above. For example, a signal may be only sent when the cap is removed, and the monitor may be in a low power state until the cap is detected as removed, on the basis that no medication is being validly dispensed until the cap is removed.

Further, in place of switches, alternative sensors may be used to detect movement of the cover or the tether. These could include, for example, optical, pressure sensors, magnetic sensors or any other suitable device.

Adherence monitor 8 as described includes an ECM, with the ECM being adapted to monitor and/or manipulate and/or store and/or transmit all adherence data gathered, relating to the patient usage of the medicament delivery device. The ECM may be a suitable microprocessor device.

The use of ECM's, in conjunction with adherence monitors for medicament delivery devices, are well known, and it is not intended therefore to describe them in any significant detail herein. For example, these systems are in general terms in commercial use in products available from the present applicant and related companies, as well as disclosed in the applicant's prior patent filings, for example those incorporated by reference herein. An example of an adherence monitor, used in conjunction with an ECM and/or transmitter can be found in U.S. Pat. No. 8,424,517 and US Patent Publication No. 2014/0000598, both by the present applicant.

The ECM is powered by a battery, and either a rechargeable or replaceable battery may be used. The ECM and/or the adherence monitor may be alternatively be powered by any suitable alternative device, for example a kinetic charger, or by solar power.

The ECM stores and transmits the adherence data gathered, so that analysis can determine if the user has used the inhaler correctly and/or incorrectly. The inhaler use logs generated in the adherence monitor are uploaded into smartphone application, a PC or a central communication hub, and through those into a web based server. In some embodiments, the inhaler use logs may also be uploaded from the adherence monitor directly into a web based server.

Adherence monitor 8 includes a memory. In some embodiments, a volatile type computer memory, including for example RAM, DRAM, SRAM, may be used. In such instances, the adherence monitor may continually transmit information to the computing device external to the adherence monitor or medicament delivery device. In other embodiments non-volatile memory formats may be used, including for example ROM, EEPROM, flash memory, ferroelectric RAM (F-RAM), optical and magnetic computer memory storage devices, and others.

The adherence monitor 8 also includes indicators, such as LED 25 to indicate an event and/or to alert the user if the ECM determines that the user has used the inhaler correctly and/or incorrectly. The indicators may be utilised to alert the user if they have attempted to dispense a dose of medicament with the cap 7 still attached. Alternatively, the indicators may be used to alert if medication has not been dispensed within certain timeframe, e.g. every 12h or 24h.

The indicators may be in the form of one or more LEDs as illustrated, or in the form of some other visual and/or audio and/or vibrational indicator. Adherence monitor 8 also includes a multi-function user button for monitoring and controlling several aspects of operation. For example, pushing the button once may result in a green light showing if the adherence monitor 8 is fitted to the inhaler 1 correctly, and in normal working order. Conversely, a red light may indicate a problem. Pushing the button twice may provide for another aspect of the adherence monitor to be checked or reported, and furthermore pushing and holding the button may result in yet another function or check being done.

The adherence monitor 8 may also include user interface enabling the user to access data recorded or received by the adherence monitor and also change the settings of the adherence monitor (for example, date/time, visual/audio alert settings). The user interface may also be used to access any data received (or transmitted) by the adherence monitor or to control the upload of the data from the adherence monitor to an external electronic device.

The embodiments of the adherence monitor 8 and/or the ECM described herein may be able to monitor for any type of non-dose counting information relating to the operation of the inhaler 1, and/or patient usage of the inhaler 1. For example, the ECM may include a real time clock (or be in electronic communication with one) to enable the adherence monitor 8 to record a date and time for each dose of medicament dispensed. The ECM may be calibrated to compare the actual doses dispensed against the table of pre-set dosage times and, if the dose is not dispensed at the pre-set time, alert the user that a dose is due.

Furthermore, and for example only, adherence monitor 8 and/or the ECM may also be able to monitor criteria such as geographical location, temperature, humidity, the orientation of the inhaler 1, the condition of the medicament, the amount of medicament left, the condition of the battery or whether it is installed, the flow or pressure of the user's inhalation, an audio sensor for detecting inhalation or for determining if the main body portion has been rotated with respect to the base portion, and so on. To this effect, the ECM may include an audio or optical inhalation sensor, thermistor sensor or accelerometer, or be connected to a GPS (e.g. the adherence data from the smartphone paired with the adherence monitor 8 may be matched with the GPS data relating to the location of adherence events received by the smartphone).

Adherence monitor 8 may also include a communication device for transmitting the adherence data. In one embodiment, this may be a USB port located on the housing 9 of the adherence monitor 8. Any other suitable wired connections or ports may be used.

Alternatively and/or additionally, the adherence monitor 8 and/or ECM may be provided with a wireless transmitter and/or a wireless transceiver e.g. Bluetooth Low Energy® module to be able to transmit and/or receive data respectively. Any other suitable wireless technology known in the art may be used, including for example Wi-Fi (IEEE 802.11), Bluetooth®, other radio frequencies, Infra-Red (IR), GSM, CDMA, GPRS, 3G, 4G, W-CDMA, EDGE or DCDMA200 or similar.

The data may be transmitted to a remote computer server or to an adjacent electronic device such as a smart phone or electronic tablet. The adherence monitor may be paired with a smartphone loaded with a software application which allows the smartphone to access, process, and/or present the data collected by the adherence monitor. The smartphone may be configured to transfer the data obtained from the adherence monitor to a web services platform. The data may be transmitted in real time, manually or at predetermined set times.

It will accordingly be understood that a processor running suitable software must be provided in order to implement some embodiments. Such a control device may be provided, for example, using the ECM within the device, and the data at that stage being processed in accordance with some embodiments to determine cap on or cap off. The data, in a raw or part processed state, may be sent to a remote but nearby device, such as a smartphone, tablet or computer. This nearby device may carry out the required processing to determine cap on or cap off. In other implementations, the data is processed by a remote server or other system. For example, this may be a proprietary server provided to carry out these (and other) functions relating to adherence monitoring, or it could be a physician-operated system specific to that physician's patients. The term “control device” is intended to encompass any suitable processing, wherever located, including these options. Some embodiments have been described in the context of a removable adherence monitoring device. However, it is envisaged that some embodiments could be fully integrated within an inhalation device, rather than a removable device.

While the embodiments described above are currently preferred, it will be appreciated that a wide range of other variations might also be made within the general spirit and scope of some embodiments.

For example other types of switches may be used to detect the absence or presence of the cap on the mouthpiece of the inhaler: e.g. any type of carbon pill, tack switch or detector switch may be used. Some examples of suitable switches include: http://www.diqikev.co.nz/product-detail/en/ESE-13V01D/P13356SCT-ND/822292; http://www.digikev.co.nz/product-detail/en/ESE-16J001/P14266SCT-ND/2193025; or http://www.digikev.co.nz/product-detail/en/FSM4JSMATR/450-1759-1-ND/2271638.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

All patent and other references noted in the specification, including websites, are hereby incorporated by reference.

1. An adherence monitor for use with a medicament inhaler, the inhaler including a body, a mouthpiece for operatively inhaling the medicament, and a cap for the mouthpiece, the adherence monitor comprising:
at least two sensors associated with the cap, the at least two sensors being configured so that removal of the cap triggers both of the sensors.

2. The adherence monitor according to claim 1, further including a processor-based controller for receiving data from the sensors, the processor based controller being configured to determine that a cap removal event has occurred only if the at least two sensors have both been triggered.

3. The adherence monitor according to claim 2, wherein the cap removal event is detected only if a second of the at least two sensors is triggered within a predetermined time delay after a first of the at least two sensors is triggered.

4. The adherence monitor according to claim 1, wherein the adherence monitor is adapted for use with a cap that is tethered to the body of the medicament inhaler by a tether.

5. The adhercnce monitor according to claim 4, wherein at least one of the at least two sensors is a switch mechanically triggered by operation of the tether, so as to generate or interrupt an electrical signal to a processor-based controller.

6. The adherence monitor according to claim 1, wherein the adherence monitor is integrated into a medicament inhaler.

7. A method for detecting removal of a cap from a medicament inhaler, the medicament inhaler including at least a body, a mouthpiece for operatively inhaling the medicament, and a cap for the mouthpiece, the method:
providing at least two sensors, each of the at least two sensors being arranged to be triggered by removal of the cap;
determining at a control device whether both sensors have been triggered; and
if both sensors have been triggered, thereby determining that a cap removal event has occurred.

8. The method according to claim 7, wherein the cap removal event is detected only if a second of the at least two sensors is triggered within a predetermined time delay after a first of the at least two sensors is triggered.

9. The method according to claim 7. wherein the medicament inhaler has a cap is tethered to the body by a tether.

10. The method according to claim 9, wherein at least one of the at least two sensors is a switch mechanically triggered by the operation of the tether, so as to generate or interrupt an electrical signal to a processor-based controller.

11. The adherence monitor according to claim 2, wherein the adherence monitor is adapted for use with a cap that is tethered to the body of the medicament inhaler by a tether.

12. The adherence monitor according to claim 3, wherein the adherence monitor is adapted for use with a cap that is tethered to the body of the medicament inhaler by a tether.

13. The adherence monitor according to claim 2, wherein the adherence monitor is integrated into a medicament inhaler.

14. The adherence monitor according to claim 3, wherein the adherence monitor is integrated into a medicament inhaler.

15. The adherence monitor according to claim 4, wherein the adherence monitor is integrated into a medicament inhaler.

16. The adherence monitor according to claim 5, wherein the adherence monitor is integrated into a medicament inhaler.

17. The method according to claim 8, wherein the medicament inhaler has a cap that is tethered to the body by a tether.
